(19) United States
(12) Patent Application Publication
(10) Pub. No.: US 2006/0228813 A1
(43) Pub. Date: Oct. 12, 2006

(54) Method for immobilizing biomolecules on metal oxide substrates

(76) Inventors: Ying Wu, Njimegen (NL); Robby Ruijtenbeek, Utrecht (NL)

Correspondence Address: Craig J Arnold;Amster Rothstein & Ebenstein, 90 Park Avenue, New York, NY, 10016, US

(21) Appl. No.: 10/549,824

(22) PCT Filed: Apr. 6, 2004
(86) PCT No.: PCT/EP04/03668

§ 371 Date: Sep. 20, 2005

(30) Foreign Application Priority Data
Apr. 10, 2003 (EP) 03447091.4

(51) Int. Cl. C12Q 1/68; C12M 1/34; B32B 15/02; G01N 33/551

(52) U.S. Cl. 436/524; 435/6; 435/287.2; 428/404

(57) ABSTRACT

The present invention relates to an improved method for providing biomolecules on a metal oxide substrate. In another aspect, the invention relates to a metal oxide substrate, having a surface that is coated with a polymer, said substrate having biomolecules immobilised thereon, wherein said biomolecules are immobilized on said coated surface of said substrate by covalent binding, by means of electromagnetic irradiation. In another aspect, the invention relates to the use of a metal oxide substrate according to the invention for performing probe-based assays.

### FIELD OF THE INVENTION

The present invention relates to the field of molecular biology. In a first aspect, the present invention relates to a method for preparing a metal oxide substrate provided with biomolecules. In another aspect the present invention relates to metal oxide substrates having biomolecules immobilised thereon. In a further aspect, the present invention relates to the use of a metal oxide substrate according to the present invention for performing probe-based assays.

### BACKGROUND OF THE INVENTION

Microarrays comprising a metal oxide substrate loaded with biomolecules are known in the art and are useful for performing probe-based assays, including gene expression profiling, mutation detection, hybridisation, immunoassays, receptor/ligand assays, or for separating other substances from mixtures by hybridising, binding or interacting with those other substances.

A particular advantageous type of metal oxide substrate for use in arrays for probe-based assays is described in WO 99/02266 and relates to a porous substrate, which is electrochemically manufactured. These substrates can be manufactured cheaply through electrochemical etching of a metal sheet. Such substrates have through-going, oriented channels with well-controlled diameter and advantageous chemical surface properties. The metal oxide substrates having such through-going channels provide more accurate and reliable detection results, and reduced background interference, when used in probe-based assays.

Several methods have been disclosed for preparing metal oxide substrates loaded with biomolecules. One method comprises the steps of activating the surface of the substrate by means of a silanating agent comprising an amine group; and subsequently loading the substrate by attaching biomolecules to the silanized or activated surface. Such a method is known, e.g. from WO 99/002266, in which method aluminium oxide substrates may be activated using 3-aminopropyl triethoxysilane (APS) after which oligonucleotide probes are covalently coupled to the activated substrates.

Improvements in the preparation of loaded metal oxide substrates are disclosed in for example WO 01/12846 wherein increased specificity for the envisaged interactions is obtained by treating the loaded support with an acidic solution. As such, unloaded amino-groups, which may be present and result in unwanted interactions are removed from the substrate, without affecting the loaded part of the surface.

According to the above-described methods, the surface of the substrate is activated prior to loading thereof with biomolecules to obtain a loaded surface. Thereto, the substrate surface is (poly)-functional or able of becoming (poly)-functionalised or activated with reactive groups capable of forming a covalent bond with the biomolecule(s) to be immobilized. Although proven to be efficient, the methods as described above often involve the loading of the activated surfaces with biomolecules having attached thereon reactive functional groups such as amino groups, aldehyde groups, thiol groups, or biotin compounds which are generally expensive and may suffer instability.

As will be appreciated in the art, there is a continuous need for further improved methods and devices suitable for probe-based microarray analysis.

The above-mentioned disadvantages can be overcome by the use of a polymer for coating of substrates, designed to electrostatically bind negatively charged oligonucleotides. Polymer coating of substrates is well known in the art related to glass substrates. Such polymer coated glass substrates are widely commercially available.

In contrast to glass substrates, metal oxide substrates in general, and porous metal oxide substrates in particular, are known to be highly inert and due to the porosity have a low surface adsorption. In addition, they are inherently hydrophilic. As a consequence thereof, it is difficult to provide a polymer coat for stable attachment of biomolecules onto such metal oxide substrates.

It is thus an object of the present invention to provide improved metal oxide substrates.

It is a general object of the present invention to provide an improved method for preparing metal oxide substrates having biomolecules immobilised thereon. It is in particular an object to provide an improved method for immobilising biomolecules on a metal oxide substrate, wherein the biomolecules to be immobilized do not need to be pre-activated with reactive functional groups.

In addition, it is another object of the present invention to provide an improved metal oxide substrate having biomolecules immobilised thereon.

### SUMMARY

The present invention relates to an improved metal oxide substrate having biomolecules immobilised thereon and a method for preparing such substrate.

In a first aspect the present invention relates to an improved method for providing biomolecules on a metal oxide substrate comprising the steps of:
a) coating said substrate with a polymer by bringing said substrate into contact with a solution comprising said polymer such that the polymer in said solution is able to form a coating on a surface of said substrate,
b) deposing said biomolecules onto the substrate obtained in step a) by bringing said biomolecules into contact with said substrate, and
c) immobilizing said biomolecules onto the substrate obtained in step a) by covalently binding said biomolecules to said substrate by means of electromagnetic irradiation.

In a preferred embodiment, the metal oxide substrate prepared according to the present method is a porous metal oxide substrate, preferably an aluminium oxide substrate, having oriented through-going channels, preferably an electrochemically manufactured porous metal oxide substrate.

An important characteristic of the present method is the use of a polymer for coating the substrate. It has now been found that a polymer, and in particular a polypeptide can be adsorbed strongly onto a metal oxide surface and is particularly suitable for coating the substrate. Coating of the substrate with such polymer enables the easy and rapid attachment of biomolecules to the coated substrate.

Another important characteristic of the present method consists in the provision of the polymer molecules in the coating to be mutually covalently bound. This is achieved by UV radiation. As a result thereof, a very stable substrate is obtained. Such UV irradiation further provides real covalent bonds between the biomolecules and the coated substrate.

In a second aspect the invention relates to a metal oxide substrate prepared according to the present method, having a surface that is coated with a polymer, said substrate having biomolecules immobilised thereon, wherein said biomolecules are immobilised on said substrate by covalent binding by means of electromagnetic irradiation.

In a preferred embodiment, said metal oxide substrate is a porous metal oxide substrate, preferably an aluminium oxide substrate, having oriented through-going channels, and preferably an electrochemically manufactured porous metal oxide substrate.

The metal oxide substrates according to the invention are particularly useful in probe-based assays. Those skilled in the art will immediately recognize the many other effects and advantages of the present method and metal oxide substrates and the numerous possibilities for end uses of the present invention from the detailed description and examples provided below.

### DETAILED DESCRIPTION OF THE INVENTION

The present invention has surprisingly provided a cost-effective method that enables rapid and easy immobilisation of biomolecules on an inert metal oxide substrate.

In a first embodiment, the present invention relates to a method for providing biomolecules on a metal oxide substrate comprising the steps of:
a) coating said substrate with a polymer by bringing said substrate into contact with a solution comprising said polymer such that the polymer in said solution is able to form a coating on a surface of said substrate,
b) deposing said biomolecules onto the substrate obtained in step a) by bringing said biomolecules into contact with said substrate, and
c) immobilizing said biomolecules onto the substrate obtained in step a) by covalently binding said biomolecules to said substrate by means of electromagnetic irradiation.

A number of materials suitable for use as substrates in the present invention have been described in the art. In view of strength and rigidity, a metal or a ceramic metal oxide may be used. As a metal, for example, a porous substrate of stainless steel (sintered metal) may be used. For applications not requiring heat resistance, a porous substrate of an organic polymer may also be used if it is rigid. Above all, in view of heat resistance and chemical resistance, a metal oxide may be used.

Materials particularly suitable for use as substrates in the present invention include porous metal oxide substrates known in the art. The term “porous substrate” as used in the present specification refers to a substrate possessing or full of pores, wherein the term “pore” refers to a minute opening or microchannel by which matter may be either absorbed or passed through. Particularly, where the pores allow passing through of matter, the substrate is likely to be permeable.

Metal oxides as employed within the present invention provide a substrate having both a high channel density and a high porosity, allowing high-density arrays comprising different target molecules per unit of the surface for sample application. In addition, metal oxides are highly transparent for visible light. Metal oxides are relatively cheap substrates that do not require the use of any typical microfabrication technology and, that offer an improved control over the liquid distribution over the surface of the substrate, such as electrochemically manufactured metal oxide membrane. Metal oxide membranes having through-going, oriented channels may be manufactured through electrochemical etching of a metal sheet.

Metal oxide substrates or membranes as employed in the methods of the present invention may be anodic oxide films. As well known in the art, an aluminium metal substrate may be anodised in an electrolyte to produce an anodic oxide film. The anodization process results in a system of larger pores extending from one face and interconnects with a system of smaller pores extending from the other face. Pore size is determined by the minimum diameters of the smaller pores, while flow rates are determined largely by the length of the smaller pores, which can be made very short. Accordingly, such membranes may have oriented through-going partially branched channels with well-controlled diameter and useful chemical surface properties. Advantageously, such membranes are transparent, especially if wet, which allows for assays using various optical techniques. WO 99/02266 which discloses the Anopore™ porous substrate is exemplary in this respect, and is specifically incorporated by reference in the present invention.

Particularly useful porous substrates as employed in the methods described in the present specification are 3-dimensional substrates, which allow pressurized movement of fluid, e.g. the sample solution, through its structure. As such, particularly useful porous substrates as employed in the present methods possess a permeable and flow-through nature. In contrast with two-dimensional substrates, 3-dimensional substrates or microarrays as employed in the methods as described herein give significantly reduced hybridisation times and increased signal and signal-to-noise ratios. Further, a positive or negative pressure may be applied to the arrays in order to pump the sample solution dynamically up and down through the substrate pores. Said dynamical pumping allows immediate removal and ability to perform real-time detection of generated products from a reaction which takes place within the pores of the substrate by fast binding of said generated products to the substrate pore walls.

As used herein, the term “surface” of said substrate refers to the outer and/or the inner surface of the substrate. The surface of the substrate on which a biomolecule is immobilised may be an external surface or an internal surface of the porous substrate, or a combination of both. Particularly where the substrate is porous, the molecule is likely to be attached to an internal surface. Biomolecules may be immobilised on the complete surface or on specified regions of the surface.

As used herein the terms “coating” or “providing a coating” refer to the process of applying a thin layer of a substance on the substrate. As used herein these terms may refer to providing a substance over the complete surface or over only a part of the surface of the substrate, whereby the surface may include the inner surface as well as the outer surface, or both. Coating of a substrate surface typically provides an activated substrate surface. Suitable substances used in the present invention to obtain activated substrate surfaces are polymers. The term “activated” as used in the present invention refers to the presence of reactive groups on the substrate surface capable of reacting with a modified or unmodified target biopolymer to cause the target biopolymer to be immobilized on the surface, such as by covalent or non-covalent attachment.

The term “deposing” is used as a synonym for “loading” and refers to the mere deposition of (bio)molecules onto the activated surface of a substrate or a part thereof, i.e. without actual bonding of said biomolecules to the activated surface such as formation of chemical or covalent bonds.

The term “immobilizing” as used in the present specification refers to the attachment or adherence of one or more biomolecules to the activated surface of a porous substrate including attachment or adherence to the activated inner surface of said substrate.

The terms “covalently binding”, “covalently attaching” or “cross-linking” are used herein as synonyms and refer to the formation of real chemical bonds between molecules. When a biomolecule is covalently attached to a substrate, this means that this molecule is attached by means of covalent chemical linkage. The term “chemical bonds” and “covalent bonds” are used herein as synonyms.

The term “polymer” as used herein refers to molecules consisting of at least two repeated chemical units joined together. The term polymer as used herein implies positively, negatively or neutrally charged polymers as well as co-polymers, such as by way of example and not limitation, polytrimethylaminomethylmethacrylate, polydimethylaminomethylmethacrylate, polymethylaminomethylmethacrylate and polyaminomethylmethacrylate. Polymers according to the invention may comprise multiple amide functional groups, such as in “polypeptides”, multiple cationic (i.e. positively charged) functional groups, such as in “polyamines”, or a combination of multiple amide and multiple cationic functional groups, such as in “polyamine polypeptides”. Substrates according to the present invention may be coated with poly-cationic substances of which non-limiting examples include protamine sulfate grade X, protamine chloride grade V, protamine phosphate grade X, poly-L-lysine hydrobromide, spermidine phosphate salt, spermidine diphosphate salt and Protosan G113 and CL113.

The term “polypeptide” as used herein refers to a polymer of amino acids, which may include positively charged, negatively charged as well as neutral amino acids, including polymers of amino acids having different enantiomeric forms, i.e. L as well as D forms of the amino acids. Polypeptides are chains of amino acids held together by amide bonds. Examples of suitable polypeptides for use in the present invention comprise but are not limited to poly-aspartate, poly-glutamate, poly-cysteine, poly-serine, poly-methionine, poly-arginine, poly-histidine, poly-tryptophane, poly-alanine, poly-lysine, poly-leucine, poly-isoleucine, poly-tyrosine, poly-valine, poly-glycine, poly-proline, poly-phenylalanine, poly-threonine; polymers of other natural amino acids (e.g., ornitine); polymers of non-natural amino acids (e.g., beta-amino acids, homo-lysine, NH₂—CH—(CH₂)ₓ—NR₃—COOH); and derivatives (e.g., N-methyl lysine, phosphotyrosine) and mixtures (e.g., [Pro-Lys-Pro-homoLys-]x) thereof.

The term “polyamine” as used herein refers to amine-containing polymers, i.e. polymers of molecules consisting of repeated chemical units having —NR₄, functional groups including primary, secondary, tertiary, and quaternary amines. Examples of polyamines suitable for use in the present invention comprise but are not limited to polyethyleneimine, tetraethylenepentamine, ethylenediamine, diethylenetriamine, triethylenetetramine, pentaethylenehexamine, hexamethylenediamine, phenylenediamine, poly(N-methyl-vinylamine), poly(allylamine), or the like.

The term “polyamine polypeptide” as used herein refers to polymers of molecules consisting of repeated chemical units having amide functional groups and amine functional groups. Example of suitable polyamine polypeptides for use in the present invention comprise but are not limited to poly-lysine, poly-ornithine, poly-arginine, and natural and non-natural derivatives or mixtures thereof.

The terms “biomolecule”, “target”, “target-molecule” and “target-biomolecule” are used interchangeably throughout the present invention and refer to molecules immobilized on a substrate; also referred to as immobilized probes or capture probes. A wide variety of different molecules can be immobilized on the substrate of the present arrays. Similarly, the present methods are applicable to a wide variety of different molecules or targets that may be immobilized on the present substrate. A biomolecule as used in the present specification refers to any molecule, which may be attached to a substrate for the purpose of performing microarray analysis. A biomolecule further refers to a molecule that may be recognized by and/or interact with a particular analyte.

As used herein, the term “biopolymer” refers to a target molecule of interest that may be attached to a substrate according to a procedure appropriate to the structure of the biopolymer. Optionally, the biopolymer is a nucleic acid sequence, including a single stranded or double stranded polynucleotide, where the polynuclcotide may be RNA, DNA, or PNA (peptide nucleic acid, wherein the nucleotide backbone is a peptide backbone). Where the biopolymer is a protein, such as a ligand, a receptor, an antibody, cell surface protein, and the like, the probe to capture or analyte is, for example, a receptor, ligand, antibody, polynucleotide, or other biopolymer or smaller molecule capable of forming a complex with the immobilised target protein. Preferably the biopolymer is known, knowable, determinable, or otherwise identifiable.

In general, covalent binding of a biomolecule to a substrate surface requires chemical modification of the biomolecule, e.g. by means of the addition of reactive functional groups to the biomolecule. The process by which biomolecules are provided with reactive functional groups is also referred to as “activating the biomolecules”. Biomolecules may be activated by attaching to their terminal group a reactive functional group such as an amino group, an aldehyde group, a thiol group, a biotin compound, or the like. However, such functional groups are generally very expensive and often may be instable.

By providing UV irradiation for covalently binding the biomolecules on the coated or activated surface of the substrate in the method according to the present invention no special or expensive active groups need to be attached to the biomolecules for covalently immobilising these on the coated substrate. The present invention thus provides a method wherein said biomolecules are preferably not chemically modified or activated prior to attachment to the polymer-coated surface of the substrate, but are preferably directly covalently bound to the coated surface of the substrate. The use of UV irradiation for covalently binding the biomolecules on the coated surface of the substrate provides effective covalent binding of a biomolecule to the polymer coat in a rapid and easy way. In addition, the method according to the invention also provides effective covalent interlinkage of the polymers relatively to each other. The covalent bonds are of importance for providing a more stable substrate.

In another preferred embodiment the present invention relates to a method wherein the polymers are substantially adsorptively bound on the metal oxide substrate. The term “adsorptively” refers to the adherence of the polymers to the substrate without forming covalent bonds. The cross-linking step by means of electromagnetic irradiation according to the present method preferably provides only to a lower extent a covalent bonding between the metal oxide substrate and the polymer coat, and the polymer coating is thus “substantially adsorptively” bound on the metal oxide substrate. Because the adsorptive binding of the polymer to the substrate involves many ‘relative’ weak bonds the result is relative strong and irreversible binding of the polymer to the substrate. The present invention advantageously and surprisingly proves the feasibility of such type of binding on metal oxide surfaces and thus provides a novel activation method to these type of substrates. The present invention therefore provides an improved activated metal oxide substrate in addition to metal oxide substrates being activated by chemical modification by for example silanization by means of the addition to the substrate of a silane-coupling agent as well known in the art which further requires the activation of the biomolecules to be immobilised thereon. Therefore, in another preferred embodiment, the present invention provides a method wherein the metal oxide substrate does not need to be, polyfunctionalised or activated by way of chemical silanization with reactive functional groups prior to providing biomolecules on said substrate.

In a preferred embodiment, the method according to the invention comprises a first step of coating a metal oxide support by submerging said substrate in an aqueous solution comprising a polymer such that the polymer in said solution is able to form a coating on the surface of said substrate. Then the substrate is removed from the solution comprising the polymer and allowed to dry for a suitable period. Subsequently, the dried substrate may be stored, preferably for at least one week. Preferably, the concentration of the polymer used in the submerging step is between 0.0001 and 1 % w/v, and more preferably between 0.001 and 0.01 % w/v. The metal oxide substrate is preferably submerged during a reaction time between 5 minutes and 72 hours, more preferably during 60 minutes, at a temperature preferably comprised between 15° C. and 50° C., and preferably at room temperature, and at a pressure preferably below 0.2 bar. The time of drying the coated metal oxide substrate is preferably comprised between one week and four months.

Then the biomolecules are covalently bound to the coated surface of said substrate by deposing the biomolecules of the substrate and applying electromagnetic radiation. The coated metal oxide substrate is preferably irradiated with electromagnetic radiation having a wavelength ranging from about 40 nm to about 400 nm and more preferably at a wavelength of 260 nm, such that the biomolecules are covalently attached to the coated substrate. Preferably, irradiation is performed during 5 to 120 seconds, and more preferably during 40 seconds. In a preferred embodiment, the irradiation step is performed before the provided biomolecules become completely dry. If the biomolecules are completely air-dried, proper hydration is necessary to make the biomolecules spread out evenly over the entire area of the loaded surface before UV cross-linking is performed.

After this step, the substrate is heated by baking. The baking step is preferably performed for a period of less than 5 hours, and more preferably for 2 hours, at a temperature preferably comprised between 15° C. and 120° C. and more preferably at 80° C. The baking step provides improved covalent binding of biomolecules to the coated substrate and improved adsorptive binding of the coating layer onto the substrate.

A further step may comprise blocking amine groups of the polymer coating whereon biomolecules were not attached by using a blocking agent such as e.g. succinic anhydride. Succinic anhydride is a molecule with two active carboxyl groups. For instance, when using poly-L-lysine as a polymer, a condensation reaction takes place and for every succinic anhydride molecule one peptide bond is formed with the poly-L-lysine.

The surface of the metal oxide substrate is coated using a solution comprising a polymer. In a preferred embodiment, the polymer used in the method according to the present invention comprises multiple amide functional groups and/or multiple cationic functional groups. In another preferred embodiment said polymer is selected from the group comprising polypeptides, polyamines, polyamine polypeptides, co-polymers such as polytrimethylaminomethylmethacrylate, or mixtures thereof. In addition, it is to be understood that polypeptides of both enantiomers, i.e. L as well as D forms of the amino acids, may be used in accordance with the present method. More preferably, said polymer is selected from the group comprising poly-aspartate, poly-glutamate, poly-cysteine, poly-serine, poly-methionine, poly-arginine, poly-histidine, poly-tryptophane, poly-alanine, poly-lysine, poly-leucine, polyisoleucine, poly-tyrosine, poly-valine, poly-glycine, poly-proline, poly-phenylalanine, poly-threonine, and natural and non-natural derivatives or mixtures thereof.

A particularly preferred polymer is poly-L-lysine. Poly-L-lysine is capable of binding biomolecules, e.g. DNA molecules, via two modes, firstly by forming a non-covalent ionic interaction of the negatively charged phosphate groups in the DNA backbone with the positively charged primary amine side chain of poly-L-lysine; and secondly, by forming a covalent interaction via a reaction of the thymidin free radical of the DNA molecule, generated during the UV cross linking step, with this primary amine or with the secondary amide of the poly-L-lysine. As a consequence thereof, a particularly stable bond of the biomolecules to the metal oxide substrate can be obtained when using this specific polymer.

In another preferred embodiment, said metal oxide substrate is a porous metal oxide substrate. The term “porous substrate” as used herein refers to a substrate possessing or full of pores, wherein the term “pore” refers to a minute opening or microchannel by which matter may be either absorbed or passed through. Particularly, where the pores allow passing-through of matter, the substrate is likely to be permeable. According to the invention, various pore sizes may be employed. The porous substrate may be planar or have simple or complex shape.

In a preferred embodiment, the metal oxide substrate is a substrate having oriented through-going channels. Even more preferred the metal oxide substrate is a substrate having oriented through-going channels such as the one described in WO 99/02266.

More preferably, the channels are opening out on a surface for sample application and the channels in at least one area of the surface for sample application are provided with a biomolecule capable of binding to an analyte. Metal oxide substrates having such through-going channels provide more accurate and reliable detection results, and reduced background interference, when used in probe-based assays. Metal oxide substrates having through-going, oriented channels may be manufactured through electrochemical etching of a metal sheet.

The kind of metal oxide is not especially limited. Metal oxides considered are, among others, oxides of zirconium (zirconia, ZrO₂), silicium (silica, SiO₂), titanium (titania, TiO₂), tantalum (Ta₂O₅), aluminium (alumina, Al₂0₃); mullite; cordierite; zeolite or zeolite analog; as well as alloys of two or more metal oxides and doped metal oxides and alloys containing metal oxides. A metal oxide substrate according to the present invention is not glass. In a more preferred embodiment, a method as described herein is provided, wherein said metal oxide substrate is an aluminium oxide substrate.

In another preferred embodiment of the method according to the invention, biomolecules are covalently bound to the coated surface of the substrate in spots, thereby forming a (micro)array of spots.

In the present invention, biomolecules are immobilized on the substrate at a spatially predefined region, i.e. at a particular spot. The terms “predefined region” or “spot” are used interchangeably in the present specification and relate to individually, spatially addressable positions on a substrate.

A predefined region is a localized area, typically on the top-surface of the substrate which is, was, or is intended to be used to target molecule deposition. Subsequent target molecule immobilization may be on said top surface (external) or on the surface of the pores within the porous substrate (internal surface) or both.

The predefined region may have any convenient shape, e.g., circular, rectangular, elliptical, wedge-shaped, etc. A predefined region may be smaller than about 1 cm² or less than 1 mm². Usually, the regions have an area of less than 50.000 μm², more usually less than 10.000 μm², or more usually less than 100 μm² and may be less than 10 μm².

The predefined regions on the substrate are spatially arranged and laid out in precise patterns, such as rows of dots, or rows of squares, or lines to form distinct arrays. The term “microarray” as used in the present specification refers to a porous metal oxide substrate, with a matrix of target-molecules arrayed at specific positions.

The substrates of the present invention may be of any desired size, from two spots to 10⁶ spots or even more. The upper and lower limits on the size of the substrate are determined solely by the practical considerations of working with extremely small or large substrates.

For a given substrate size, the upper limit is determined only by the ability to create and detect spots in the microarray. The preferred number of spots on a microarray generally depends on the particular use to which the microarray is to be put. For example, sequencing by hybridisation will generally require large arrays, while mutation detection may require only a small array. In general, microarrays according to the present invention contain from 2 to 10,000 spots per square millimeter. A particular useful spot density is within a range of 2 to 1000 spots per mm². A more particular useful spot density is within a range of 2 to 100 spots per mm². Usually a microarray useful in the present invention has a spot density of 25 spots per mm².

Furthermore, not all spots on the microarray need to be unique. Indeed, in many applications, redundancies in the spots are desirable for the purposes of acting as internal controls.

Methods and arrays of the present invention may have incorporated the use of immobilized internal references, which may bind to reporter molecules to correct for signal errors due to variations in sample preparation. In this regard, the International patent application PCT/EP02/14426 is exemplary, and is specifically incorporated in the present invention.

In yet another embodiment of the present method said covalently attached biomolecules may comprise the same or different biomolecules. Preferably, substrates according to the invention comprise different biomolecules in different spots, allowing multi-analyte detection.

Alternatively, each spot of the array may comprise a mixture of polynucleotides of different sequences. These mixtures may comprise degenerate polynucleotides of the structure NxByNz, wherein N represents any of the four bases and varies for the polynucleotides in a given mixture, B represents any of the four bases but is the same for each of the polynucleotides in a given mixture, and x, y, z are integers. Alternatively, spots may comprise mixtures of polynucleotides that correspond to different regions of a known nucleic acid; these regions may be overlapping, adjacent, or non-adjacent. Arrays comprising these types of mixtures are useful in, for example, identifying specific nucleic acids, including those from particular pathogens or other organisms. Both types of mixtures are discussed in WO 98/31837, herewith incorporated by reference.

A preferred method to spot the surface with biomolecules applies inkjet technology. This technology allows for the accurate deposition of defined volumes of liquid. (See e.g. T. P. Theriault: DNA diagnostic systems based on novel Chem-Jet technologies, IBC Conference on Biochip Array Technologies, Washington DC, May 10, 1995).

In another embodiment of the method according to the invention the biomolecules are selected from the group comprising oligonucleotides, polynucleotides, ribonucleotides, proteins, antibodies, antigens, peptides, oligo or poly sacharides, receptors, haptens and ligands. As mentioned above, the biomolecules do not need to be chemically modified prior to loading to the coated substrate.

The methods and arrays are particularly exemplified herein in terms of nucleic acid sequences including deoxyribonucleic acids (DNA, cDNA), ribonucleic acids (RNA, mRNA, cRNA, aRNA), peptide nucleic acids (PNA) and/or fragments thereof including polynucleotides and oligonucleotides as biomolecules, immobilized on a substrate.

The immobilized molecules may be tailored to specifically bind to or hybridise with specified analyte molecules. For example, if a substrate according to the invention is used to determine expression of a particular gene from a cDNA library that has been reverse transcribed from mRNA molecules, the immobilized molecules will be constructed with a sequence complementary or otherwise capable of recognizing the gene, gene fragment or expression products of such gene or gene fragments. In this context, the nucleic acids may be derived from any biological sources including, but not limited to, human, animal, plants, bacterial, fungal, viral, environmental or other sources.

The composition of the immobilized polynucleotides is not critical. The only requirement is that they be capable of hybridising to a nucleic acid of complementary sequence, if any. For example, the polynucleotides may be composed of all natural or all synthetic nucleotide bases, or a combination of both. Non-limiting examples of modified bases suitable for use with the instant invention are described, for example, in Practical Handbook of Biochemistry and Molecular biology, G. Fasman, Ed., CRC Press, 1989, pp. 385-392. While in most instances the polynucleotides will be composed entirely of the natural bases (A, C, G, T or U), in certain circumstances the use of synthetic bases may be preferred.

The length of the immobilized biomolecules, in instances where they are nucleotides, polynucleotides, nucleic acids or similar polymers, will usually range between 5 to 1000 nucleotides, optionally 5 to 500 nucleotides, further optionally 5 to 250 nucleotides, still further optionally, 20 to 100 nucleotides. The polynucleotide, oligonucleotide or nucleic acid probes may be double or single stranded, or PCR fragments amplified from cDNA.

The methods and substrates according to the present specification are equally applicable to other types of molecules. For example, one skilled in the art could easily adapt the present methods and arrays to apply to targets including for example proteins such as antibodies, antigens, peptides, oligo or poly-sacharides, receptors, haptens and ligands, drugs, toxins, liposomes and more.

In another embodiment, the invention relates to a metal oxide substrate, which is obtainable according to the method of the present invention, having a surface that is coated with a polymer, preferably with a polypeptide, and even more preferred with poly-L-lysine, said substrate having biomolecules immobilised thereon, wherein said biomolecules are immobilised on said substrate by covalent binding by means of electromagnetic irradiation. In a preferred embodiment said metal oxide substrate is a porous metal oxide substrate. In a more preferred embodiment, said porous metal oxide substrate has oriented through-going channels. In an even more preferred embodiment, the metal oxide substrate is a porous aluminium oxide substrate, having oriented through-going channels.

In another embodiment, the invention relates to a metal oxide substrate, having a surface that is coated with a polymer, said substrate having biomolecules immobilised thereon, wherein said biomolecules are immobilised on said substrate by covalent binding by means of electromagnetic irradiation. In a preferred embodiment the invention relates to a metal oxide substrate, having a surface that is coated with polypeptides, said substrate having biomolecules immobilised thereon, wherein said biomolecules are immobilised on said substrate by covalent binding by means of electromagnetic irradiation. In a more preferred embodiment, the invention relates to a metal oxide substrate, having a surface that is coated with poly-L-lysine, said substrate having biomolecules immobilised thereon, wherein said biomolecules are immobilised on said substrate by covalent binding by means of electromagnetic irradiation. In a preferred embodiment said metal oxide substrate is a porous metal oxide substrate. In a more preferred embodiment, said porous metal oxide substrate has oriented through-going channels. In an even more preferred embodiment, the metal oxide substrate is a porous aluminium oxide substrate, having oriented through-going channels.

In another embodiment, the invention provides an aluminium oxide substrate, having a surface that is coated with a polymer, said substrate having biomolecules immobilised thereon, wherein said biomolecules are immobilised on said substrate by covalent binding by means of electromagnetic irradiation. In a preferred embodiment the invention relates to aluminium oxide substrate, having a surface that is coated with polypeptides, said substrate having biomolecules immobilised thereon, wherein said biomolecules are immobilised on said substrate by covalent binding by means of electromagnetic irradiation. In a more preferred embodiment, the invention relates to an aluminium oxide substrate, having a surface that is coated with poly-L-lysine, said substrate having biomolecules immobilised thereon, wherein said biomolecules are immobilised on said substrate by covalent binding by means of electromagnetic irradiation. In a preferred embodiment said aluminium oxide substrate is a porous aluminium oxide substrate. In a more preferred embodiment, said porous aluminium oxide substrate has oriented through-going channels.

According to a particularly preferred embodiment the invention thus provides an aluminium oxide porous substrate coated with poly-L-lysine. Because of the advantageous characteristics of a porous aluminium oxide substrate and a poly-L-lysine coating, as explained above, specific combination of such substrate and such coating is particularly preferred. Since aluminium oxide is very hydrolysable and degradable by aqueous solutions having high or low pH and since the poly-L-lysine solution is of basic pH, coating of an aluminium oxide substrate with poly-L-lysine is not obvious. The present invention provides a method that is capable of modifying and coating the aluminium oxide substrate with poly-L-lysine despite the basic character of this polymer.

Metal oxide substrates according to any of the embodiments of the present invention, are very useful for performing gene expression analyses, for example in probe-based assays. Probe-based assays comprise for example nucleic acid hybridisation assays and immunological assays, sequencing by hybridisation, receptor/ligand assays and the like.

The present invention therefore also relates to a method for performing probe-based assays, comprising contacting a sample comprising an analyte to a metal oxide substrate having biomolecules immobilised thereon according to any of the embodiments of the present invention; incubating said sample with said substrate under conditions suitable for allowing binding of said analyte in said sample to said biomolecules immobilised on said substrate; and detecting the binding of said analyte in said sample to said biomolecule immobilised on said substrate.

As used herein, the term “analyte” or “analyte molecule”, “analyte nucleic acid” and “analyte sequence” are used interchangeably. An “analyte” is defined herein as a substance in a mixture that may be detected because of its capability to interact specifically with a selected reagent, e.g. biomolecules spotted on a substrate, capable of reacting with the analyte. The term “analyte” refers to a nucleic acid sequence, the presence or absence of which is desired to be detected in a sample. Analyte nucleic acid can be single-stranded or double-stranded. Additionally, the analyte nucleic acid may be nucleic acid in any form most notably DNA, RNA, PNA, including fragments thereof.

As used herein, the term “sample” refers to a substance that is being assayed for the presence of one or more analyte molecules of interest such as e.g. nucleic acids. The nucleic acid or nucleic acids of interest may be present in a mixture of other nucleic acids. A sample, containing nucleic acids of interest, may be obtained in numerous ways known in the art.

Virtually any sample may be analysed using the method according to the present specification including cell lysates, purified genomic DNA, body fluids such as from a human or animal, clinical samples, food samples, etc. Usually, the sample is a biological or a biochemical sample. The term “biological sample,” as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, cerebrospinal fluid, blood, blood fractions such as serum including fetal serum (e.g., SFC) and plasma, blood cells (e.g., white cells), tissue or fine needle biopsy samples, urine, peritoneal fluid, and pleural fluid, or cells there from. Biological samples may also include sections of tissues such as frozen sections taken for histological purposes. The sample can be, for example, also a physiological sample.

Samples may be analysed directly or they may be subject to some preparation prior to application on a substrate according to this invention. Non-limiting examples of said preparation include suspension/dilution of the sample in water or an appropriate buffer or removal of cellular debris, e.g. by centrifugation, or selection of particular fractions of the sample before analysis. Nucleic acid samples, for example, are typically isolated prior to assay and, in some embodiments, subjected to procedures, such as reverse transcription and/or amplification (e.g., polymerase chain reaction, PCR) to increase the concentration of all sample nucleic acids (e.g., using random primers) or of specific types of nucleic acids (e.g., using polynucleotide-thymidylate to amplify messenger RNA or gene-specific primers to amplify specific gene sequences). The amplification method set out in WO 99/43850 may also be used in the present invention.

In probe-based assays, a sample that comprises an analyte is contacted with a substrate provided with biomolecules prepared according to the invention. The analyte is subsequently allowed to bind to the biomolecule that is covalently attached to the surface of the substrate. Detection of binding can be performed by (1) adding a detection means, for example a substance capable of binding to the analyte, which substance is provided with a label, (2) allowing the detection means to bind to the complex of the analyte and the biomolecule, and (3) determining whether the label is present at the position where the biomolecule was attached. Alternatively, the analyte may already have been provided with a label, in which case binding to the biomolecule can be detected directly, without the addition of a detection means.

In an example, a DNA-containing sample is subjected to extraction to separate mRNA or genomic DNA, from which cDNA or target DNA is obtained. The cDNA or target DNA is labelled with a fluorescence indicator to give a labelled target DNA fragment (which may be a labelled RNA fragment). The labelled target DNA fragment is then hybridised with the oligonucleotide or polynucleotide of a substrate according to the present invention to obtain a hybridised substrate. The hybridised substrate is scanned by fluorometry in a known DNA scanning fluorometric apparatus, to give a graphical representation of the positions where the hybridised DNA fragments are present.

When a substrate according to the invention is used as a tool to obtain DNA sequence information, a large array of areas is provided, each area comprising as a first binding substance an oligonucleotide probe of a different base-pair sequence. If a sample containing DNA or RNA fragments with a (partly) unknown sequence is brought into contact with said substrate a specific hybridisation pattern may occur, from which pattern the sequence information of the DNA/RNA can be derived. Such “sequencing by hybridisation” methods are well known in the art (see e.g. Fodor, S. P. A. et al. (1992), Science 251, 767-773 and Southern, E. M. et al. (1994) Nucleic Acids Res. 22, 1368-1373).

A substrate according to the present invention may also be used to screen a biological specimen, such as blood, for a large number of analytes. An array may consist of areas comprising oligonucleotide probes specific for, for example, E. coli, S. aureus, S. pneumoniae etc. If a biological sample is brought into contact with the substrate, the resulting hybridisation pattern can be read e.g. using a CCD camera in combination with an appropriate optical marker. Apart from screening for bacteria, the substrate is suitable for the detection of viruses, as well as the classification of different subtypes of, for example, HIV- and HCV viruses, etc. Virus classification may be essential to determine potential drug resistance. In general it requires the ability to detect single point mutations in the virus RNA.

A substrate according to the invention is also suitable for performing sandwich immunoassays. In that case, it is preferred that a second antibody is used for binding to bound analyte, said second antibody for each of the analyte being recognised by a third labelled antibody. This may be achieved if the second and third antibodies are derived from different species and the third antibody is raised against antibodies of the other species. Thus it is avoided to label the second antibody for each particular analyte.

A substrate according to the invention is also suited for performing “pepscans” as disclosed in Geysen et al., Proc. Natl. Acad. Sci. USA 81:3998-4002 (1984). In that case the first binding substances that are attached to the different areas of the substrate constitute different sequences of aminoacids. If the substrate is brought into contact with a liquid that contains a particular analyte, a reaction pattern may occur representing the specific affinity of the analyte for the different amino acid sequences.

Examples of analytes which may bind to biomolecules provided on a metal oxide substrate according to the present invention include, but are not limited to, antibodies including monoclonal antibodies polyclonal antibodies, purified antibodies, synthetic antibodies, antisera reactive with specific antigenic determinants (such as viruses, cells or other materials), proteins, peptides, polypeptides, enzyme binding sites, cell membrane receptors, lipids, proteolipids, drugs, polynucleotides, oligonucleotides, sugars, polysaccharides, cells, cellular membranes and organelles, nucleic acids including deoxyribonucleic acids (DNA), ribonucleic acids (RNA), and peptide nucleic acids (PNA) or any combination thereof; cofactors, lectins, metabolites, enzyme substrates, metal ions and metal chelates.

The present invention also relates to a kit of parts comprising a metal oxide substrate according to any of the embodiments of the present invention, further comprising a detection means for determining whether binding has occurred between the biomolecules and an analyte. Preferably, such detection means is a substance capable of binding to the analyte and being provided with a label. Such label is in particular useful, if it is capable of inducing a colour reaction and/or capable of bio- or chemo- or photoluminescence.

In accordance to the present invention, radio-isotope (RI) label or a non-RI label may be used. Preferably a non-RI label is utilized. Examples of non-RI labels include fluorescence label, biotin label, and chemical luminescence label. The fluorescence label is most preferably employed. Examples of the fluorescence labels include cyanine dyes (e.g., Cy3 and Cy5), fluorescein isothiocyanate (FITC), rhodamine 6G reagent, N-acetoxy-N-acetyl-aminofluorene (AAF), and AAIF (iodide derivative of AAF). The analyte labelled with different fluorescence indicators can be simultaneously analysed, if the fluorescence indicators have fluorescence spectrum of different peaks.

The invention is further illustrated by the following non-limiting examples.

### EXAMPLES

### Example 1

### Preparation of a Metal Oxide Substrate According to the Present Method

This example illustrates the preparation of a porous aluminium oxide according to the method of the present invention.

A porous aluminium oxide substrate is submerged with a poly-L-lysine solution having a concentration of 0.1% w/v for 1 hour. Subsequently, the poly-L-lysine solution is discarded and the substrate is washed with filtered HPLC-H₂O. The substrate is then dried in an oven at 37° C. for approximately 1 hour at vacuum pressure. After this step the substrate is stored at room temperature for approximately at least one week before further use. After the storage period, biomolecules are spotted on the substrate and cross-linked to the substrate by means of UV irradiation. Subsequently, the substrate is baked in an oven at 80° C. for 2 hours. Following the baking step, unloaded amine groups of the polypeptide (poly-L-lysine) on the substrate are blocked by means of a blocking agent, such as succinic anhydride dissolved in DMSO. After the blocking step the substrate is washed with DMSO and filtered HPLC-H₂O and further dried in an oven at 80° C. for approximately 10 minutes. The obtained porous aluminium oxides substrate can now be used in probe-based assays, or can be stored.

It was surprising that the Al₂O₃ metal oxide substrate was successfully coated with poly-L-Lysine. Porous Al₂O₃ metal oxide substrates as used in this experiment are not flat compared to glass substrates. Moreover, such porous Al₂O₃ metal oxide substrates are highly hydrolysable/degradable by aqueous solutions of high or low pH (poly-L-lysine solution is of basic pH). The observation of successful modification of the porous Al₂O₃ metal oxide substrate with poly-L-lysine despite the basic character of this polymer was therefore not expected.

### Example 2

### Use of a Metal Oxide Substrates According to the Present Method

Porous aluminium oxide substrates, prepared as indicated in Example 1, were used in probe-based expression analyses to analyse the expression profile of 23 human genes. The results obtained with these substrates were compared with those obtained by real-time quantitative RT-PCR, a glass cDNA array and RNA blots. Further, specificity, reproducibility and sensitivity of substrates used in this example were examined.

Preparation of an Array

Porous aluminium oxide substrates were prepared as explained in Example 1. The spotted biomolecules comprised 60-mer oligonucleotides, which were selected in both polarities using appropriate software. The substrates used in this example consisted of 120 spots in which 46 of 60-mer oligonucleotides from 23 human genes, two Cy3/Cy5 reference oligonucleotides used for microscope focusing, two negative controls (poly dA and human COT-1 DNA) and 10 exogenous alien oligonucleotides were spotted in duplicate.

Analysis of Expression Profile of 23 Human Genes

RNA was extracted from heat-treated and non-treated human T (Jurkat) cells. Heat-treated cells were incubated for four hours at 43° C. as previously described (Schena et al., PNAS 93:10614-10619, 1996).

The expression levels of 23 human genes was compared between control (HS−) and heat-treated (HS+) cells using single fluorescein labelling. Table 1 summarizes quantitative results of this experiment. Expression in 21 of the 23 human genes was detected. Differential expression (ratio >1.5) was identified in heat-treated human T cells in 12 of the 23 human genes (Table 1).

TABLE 1
Expression profile of 23 human genes monitored by substrates
according to the present invention

| | | | Normalized | |
|---|---|---|---|---|
| | | Accession | signal^{a} | |
| Blast identity | Description | no. | HS+/HS− | Ratio |
| HSP90α | stress response | X15183 | 2.50/0.29 | 8.6 |
| HSP90β | stress response | M16660 | 5.57/1.69 | 3.3 |
| Polyubiquitin | stress response | M17597 | 1.85/0.60 | 3.1 |
| TCP-1 | stress response | X52882 | 0.17/0.02 | 8.6 |
| DnaJ homolog | stress response | D13388 | 0.47/0.07 | 6.4 |
| Novel | unclassified | U56655 | 0.20/0.42 | 0.5 |
| β-actin | cytoskeletal protein | X00351 | 5.77/6.97 | 0.8 |
| PAC-1 | phosphatase of | L11329 | 0.09/0.03 | 3.4 |
| | activated cells | | | |
| PGK | Phosphoglycerate | L00160 | 0.06/0.04 | 1.5 |
| | kinase | | | |
| NF-kB1 | nuclear | M55643 | ND | ND |
| | factor-kappaB | | | |
| DUSP1 | stress response | X68277 | 0.06/0.04 | 1.4 |
| SOD1 | stress response | X02317 | 1.95/0.85 | 2.3 |
| PDLIM1 | stress response | U90878 | 1.45/1.58 | 0.9 |
| FKBP4 | stress response | M88279 | 0.244/0.18 | 1.3 |
| HSP60 | stress response | NM_002156 | 2.96/0.96 | 3.1 |
| HSP70 | stress response | NM_004134 | 0.09/0.08 | 1.1 |
| HSP40 | stress response | NM_012266 | ND | ND |
| UBE1 | stress response | M58028 | 0.31/0.21 | 1.5 |
| SmurF2 | stress response | NM_022739 | 0.1/0.09 | 1.1 |
| E2G1 | stress response | NM_003342 | 0.05/0.04 | 1.6 |
| RPL37A | ribosomal protein | L06499 | 5.48/4.84 | 1.1 |
| RPL32 | ribosomal protein | NM_000994 | 1.61/1.49 | 1.1 |
| RPL28 | ribosomal protein | NM_000991 | 0.62/0.55 | 1.1 |

^{a}The median signals of 10 alien spike genes were used for normalization.
ND not detected.

The expression level of 23 human genes was also compared between control (HS−) and heat-treated (HS+) cells using dual Cy3/Cy5 labelling. Dual labelling is common practice for gene expression in order to compare the mRNA expression level of a treated sample to a known sample. Similar expression patterns were obtained in these hybridisation experiments as compared to that of single dye fluorescein labelling.

Validation of the Results Obtained with Substrates According to the Present Invention

The expression profiling results obtained with substrates of this example were compared with those obtained by real-time quantitative RT-PCR, in glass cDNA arrays and RNA blots.

To verify expression patterns on the present substrates, real-time quantitative RT-PCR based on SYBR Green 1 assay was performed using total RNA from control (37° C.) and heat-treated (43° C.) human T cells. Real-time quantitative RT-PCR confirmed a change in expression of 8 of 12 (67%) genes as identified by arrays of this example (Table 1). Trends (up regulation or no change) identified by substrates of this example could be validated in 21 of the 23 cases. Genes with strong hybridisation signals could be validated by real-time RT-PCR.

Furthermore, the expression of the 10 genes monitored by the present arrays was compared to the expression monitored on glass cDNA arrays (Schena et al. 1996) and RNA blots (Table 2). The expression of 9 of these 10 genes could be confirmed. Differential expression of 5 heat shock genes (HSP90β, HSP90β, Polyubiquitin, TCP-1 and DnaJ homolog) was confirmed upon heat treatment.

TABLE 2
Expression profile of 23 human genes monitored by multi-platforms

| | | | | Ratio^{a} | Ratio^{a} |
|---|---|---|---|---|---|
| | | Ratio^{a} | Ratio^{a} | Glass | RNA |
| Blast ID | Accession no. | PamChip | Q-PCR | cDNA^{b} | blot^{b} |
| HSP90-α | X15183 | 8.6 | 7.9 | 5.8 | 7.2 |
| HSP90β | M16660 | 3.3 | 3.6 | 2.6 | 4.0 |
| Polyubiquitin | M17597 | 3.1 | 5.1 | 2.5 | ND |
| TCP-1 | X52882 | 8.6 | 1.9 | 2.4 | 3.8 |
| DnaJ homolog | D13388 | 6.5 | 3.0 | 4.0 | 8.1 |
| Novel | U56655 | 0.5 | 1.4 | 2.0 | 2.3 |
| β-actin | X00351 | 0.8 | 1.2 | 0.5 | 1.0 |
| PAC-1 | L11329 | 3.4 | 2.3 | 19^{c} | 71^{c} |
| PGK | L00160 | 1.5 | 1.8 | 2.6^{c} | 2.0^{c} |
| NF-kB1 | M55643 | ND | 1.6 | 3.5^{c} | 7.2^{c} |
| DUSP1 | X68277 | 1.4 | 5.2 | NA | NA |
| SOD1 | X02317 | 2.3 | 1.2 | NA | NA |
| PDLIM1 | U90878 | 0.9 | 0.8 | NA | NA |
| FKBP4 | M88279 | 1.3 | 5.9 | NA | NA |
| HSP60 | NM_002156 | 3.1 | 2.1 | NA | NA |
| HSP70 | NM_004134 | 1.1 | 1.5 | NA | NA |
| HSP40 | NM_012266 | ND | 1.5 | NA | NA |
| UBE1 | M58028 | 1.5 | 1.2 | NA | NA |
| SmurF2 | NM_022739 | 1.1 | 1.0 | NA | NA |
| E2G1 | NM_003342 | 1.6 | 1.0 | NA | NA |
| RPL37A | L06499 | 1.1 | 1.0 | NA | NA |
| RPL32 | NM_000994 | 1.1 | 1.2 | NA | NA |
| RPL28 | NM_000991 | 1.1 | 1.0 | NA | NA |

^{a}Ratio indicates heat-treated cells/control cells
^{b}Data were obtained from report of Schena et al. (1996)
^{c}Schena et al. (1996) detected an increased expression of these genes in Jurkat cells only after phorbol ester treatment. However, this treatment was not done in experiments on arrays, and real-time quantitative RT-PCR
ND not detected;
NA not available

In conclusion, the results obtained with substrates according to the present invention corresponded to those obtained with other types of arrays or detection systems.

Specificity of Substrates According to the Present Invention

To assess the specificity of gene expression on substrates of this example, negative controls (poly dA and human COT-1 DNA) and antisense oligonucleotides of 23 human genes were spotted on said substrates in duplicate. Hybridisation signals were not detectable for any of the negative controls. Furthermore, no significant signals could be detected in all antisense oligonucleotides from the 23 human genes. These results indicates that hybridisation to the selected oligonucleotides on this array in this example is very specific.

Reproducibility of Substrates According to the Present Invention

To determine the reproducibility of results obtained with the present array, four hybridisation experiments were repeated using the same two samples with fluorescein labelling under the same hybridisation conditions. The two samples comprised 5 μg of Flu-labelled amplified RNA (aRNA) from control and heat-treated cells. The raw data images obtained were analysed using appropriate software. The values of signal intensity were normalized using the mean values of median signals from the 10 exogenous alien spikes. Data from the four hybridisations with the same sample (control or heat-treated RNA) were combined so that the results from the two data sets could be examined. The average variability between arrays was below 10% CV (coefficient of variation). This indicated that results obtained with arrays according to the present invention are reproducible.

Sensitivity of Substrates According to the Present Invention

In order to assess the sensitivity of gene expression on said substrates, 6 human genes (HSP90α, HSP90β, PolyUBQ, TCP-1, Novel and β-actin) were used to determine minimum sample amount required for detection. Transcripts of these genes were generated and individually labelled with fluorescein, Cy3 or Cy5. After purifications, the concentration of individually aRNA was determined by measuring the optical density at 260 nm.

The minimum detectable amount was comprised between 1 and 4 pM, i.e. from 12×10⁶ molecules to 48×10⁶ molecules, of transcript for all tested genes using fluorescein, Cy3 or Cy5 labelling. These results are shown in Table 3.

TABLE 3
Minimum sample amount required for detection on substrates according
to the present invention

| | | Size of aRNA | input of 4 pM | | | input of 2 pM | | |
|---|---|---|---|---|---|---|---|---|
| Blast identity | Accession no. | (bp) | Flu | Cy3 | Cy5 | Flu | Cy3 | Cy5 |
| HSP90α | X15183 | 377 | + | + | + | + | + | + |
| HSP90β | M16660 | 405 | + | + | + | + | + | + |
| Polyubiquitin | M17597 | 219 | + | + | + | − | − | − |
| TCP-1 | X52882 | 357 | + | + | + | + | + | + |
| Novel | U56655 | 215 | + | + | + | − | − | − |
| β-actin | X00351 | 364 | + | + | + | + | + | + |

+, signal was detectable;
−, signal was not detectable.

In conclusion, the present example illustrates the effective use of substrates according to the present invention in probe-based assays, e.g. in gene expression analysis. Furthermore the examples illustrate that substrates according to the invention are suitable for providing specific, sensitive and reproducible data.

1. A method for providing biomolecules on a metal oxide substrate comprising the steps of:
a) coating said substrate with a polymer by bringing said substrate into contact with a solution comprising said polymer such that the polymer in said solution is able to form a coating on a surface of said substrate,
b) deposing said biomolecules onto the substrate obtained in step a) by bringing said biomolecules into contact with said substrate, and
c) immobilizing said biomolecules onto the substrate obtained in step a) by covalently binding said biomolecules to said substrate by means of electromagnetic irradiation.

2. A method according to claim 1, wherein said polymer is substantially adsorptively bound on the metal oxide substrate.

3. A method according to claim 1, wherein said polymer comprises multiple amide functional groups and/or multiple cationic functional groups.

4. A method according to claim 1, wherein said polymer is selected from the group comprising poly-aspartate, poly-glutamate, poly-cysteine, poly-serine, poly-methionine, poly-arginine, poly-histidine, poly-tryptophane, poly-alanine, poly-lysine, poly-leucine, poly-isoleucine, poly-tyrosine, poly-valine, poly-glycine, poly-proline, poly-phenylalanine, poly-threonine, polymers of other natural and non-natural amino acids and derivatives and mixtures thereof.

5. A method according to claim 4 wherein said polymer is poly-L-lysine.

6. A method according to any claim 1, wherein said metal oxide substrate is a porous metal oxide substrate.

7. A method according to claim 1, wherein said metal oxide substrate is a substrate having oriented through-going channels.

8. A method according to claim 1, wherein said metal oxide substrate is an aluminium oxide substrate.

9. A method according to claim 1, wherein the biomolecules are immobilized on the substrate in spots, thereby forming an array of spots.

10. A method according to claim 1, wherein said biomolecules comprise the same or different biomolecules.

11. A method according to claim 1, wherein said biomolecules are selected from the group comprising oligonucleotides, polynucleotides, ribonucleotides, proteins, antibodies, antigens, peptides, oligo or poly saccharides, receptors, haptens, ligands, drugs, toxins and liposomes.

12. A metal oxide substrate obtainable according to the method of claim 1, having a surface that is coated with a polymer, said substrate having biomolecules immobilized thereon, wherein said biomolecules are immobilized on said substrate by covalent binding by means of electromagnetic irradiation.

13. A metal oxide substrate according to claim 12, wherein said metal oxide substrate is a porous aluminium oxide substrate, having oriented through-going channels.

14. A metal oxide substrate, having a surface that is coated with a polymer, said substrate having biomolecules immobilized thereon, wherein said biomolecules are immobilized on said substrate by covalent binding by means of electromagnetic irradiation.

15. A metal oxide substrate according to claim 14, wherein said metal oxide substrate has a surface that is coated with a polypeptide, and preferably with poly-L-lysine.

16. A metal oxide substrate according to claim 14, wherein said metal oxide substrate is a porous aluminium oxide substrate, having oriented through-going channels.

17. An aluminium oxide substrate, having a surface that is coated with a polymer, said substrate having biomolecules immobilized thereon, wherein said biomolecules are immobilized on said substrate by covalent binding by means of electromagnetic irradiation.

18. An aluminium oxide substrate according to claim 17, wherein said substrate has a surface that is coated with a polypeptide, and preferably with poly-L-lysine.

19. An aluminium oxide substrate according to claim 17, wherein said substrate is a porous aluminium oxide substrate having oriented through-going channels.

20. A kit or parts of a kit comprising a metal oxide substrate according to claim 12, further comprising a detection means for determining whether binding has occurred between biomolecules and an analyte.

21. A kit according to claim 20, wherein the detection means is a substance capable of binding to the analyte and being provided with a label.

22. A kit according to claim 21, wherein the label is capable of inducing a color reaction and/or capable of bio-, chemi- or photoluminescence.

23. Method for performing probe-based assays, comprising the steps of:
contacting a sample comprising an analyte to a metal oxide substrate having biomolecules immobilized thereon according to claim 12;
incubating said sample with said substrate under conditions suitable for allowing binding of said analyte in said sample to said biomolecules immobilized on said substrate; and
detecting the binding of said analyte in said sample to said biomolecule immobilized on said substrate.

24. The metal oxide substrate according to claim 12, which is used for performing probe-based assays.
